SEQUENCE LISTING (10 sequences)

SEQ ID NO: 1 - DNA, 21 bp, Artificial sequence
  Synthetic oligonucleotide
ngcngacnccaaagcncngtt

SEQ ID NO: 2 - DNA, 21 bp, Artificial sequence
  Synthetic oligonucleotide
cagagcnnnggagncagcatt

SEQ ID NO: 3 - DNA, 23 bp, Artificial sequence
  Synthetic oligonucleotide
nnngcngacnccaaagcncngnn

SEQ ID NO: 4 - DNA, 21 bp, Artificial sequence
  Synthetic oligonucleotide
cagagcnnnggagncagcann

SEQ ID NO: 5 - DNA, 21 bp, Artificial sequence
  Synthetic oligonucleotide
cagagcnnnggagncagcann

SEQ ID NO: 6 - DNA, 21 bp, Artificial sequence
  Synthetic oligonucleotide
cnggacnnccagaagaacatt

SEQ ID NO: 7 - DNA, 21 bp, Artificial sequence
  Synthetic oligonucleotide
ngnncnncnggaagnccagtt

SEQ ID NO: 8 - DNA, 23 bp, Artificial sequence
  Synthetic oligonucleotide
nncnggacnnccagaagaacnnn

SEQ ID NO: 9 - DNA, 21 bp, Artificial sequence
  Synthetic oligonucleotide
ngnncnncnggaagnccagnn

SEQ ID NO: 10 - DNA, 21 bp, Artificial sequence
  Synthetic oligonucleotide
ngnncnncnggaagnccagnn

(19) United States
(12) Patent Application Publication
(10) Pub. No.: US 2009/0306177 A1
(43) Pub. Date: Dec. 10, 2009

(54) Modulation of Immunostimulatory Properties of Short Interfering Ribonucleic Acid (Sirna) by Nucleotide Modification

(75) Inventors: Eugen Uhlmann, Glashuetten (DE); Marion Jurk, Dormagen (DE); Joerg Vollmer, Duesseldorf (DE); Christian Schetter, Hilden (DE); Martin Weber, Leichlingen (DE); Ioanna Andreou, Koeln (DE); Stefan Pitsch, Zurich (CH)

Correspondence Address: WOLF GREENFIELD & SACKS, P.C., 600 ATLANTIC AVENUE, BOSTON, MA, 02210-2206, US

(73) Assignees: Coley Pharmaceutical GmbH, Duesseldorf (DE); Qiagen GmbH, Hilden (DE)

(21) Appl. No.: 11/992,073

(22) PCT Filed: Sep. 15, 2006
(86) PCT No.: PCT/IB2006/003356

§ 371 Date: Apr. 2, 2009

Related U.S. Application Data
(60) Provisional application No. 60/717,597, filed on Sep. 16, 2005.

(51) Int. Cl. A61K 48/00; C07H 21/02; A61P 37/04

(52) U.S. Cl. 514/44 A; 536/23.1

(57) ABSTRACT

Double-stranded short interfering ribonucleic acid (siRNA) are modified to reduce or eliminate their immunostimulatory effect without significantly affecting their gene silencing effect. Modified siRNA include one or more 2′ sugar modifications and, optionally, internucleotide linkages on the sense strand. Compositions containing the modified siRNA and methods of making and using the modified siRNA are disclosed. New and previously characterized siRNA can be synthesized to incorporate modifications according to the invention.

### BACKGROUND OF THE INVENTION

Ribonucleic acid (RNA) has recently been the focus of intense interest because of its newly-recognized potential as a therapeutic. It has recently been reported, for example, that certain sequence-specific double-stranded RNA, generally about 21-23 nucleotides long, can be used to silence gene expression in a selective manner, in a process called RNA interference (RNAi) or post-transcriptional gene silencing. Double-stranded RNA used for this type of RNA interference includes, in particular, so-called short interfering RNA (siRNA). Hannon GJ (2002) Nature 418:244-51. In contrast, it has also recently been reported that sequence-nonspecific double-stranded RNA can induce immunostimulatory effects, acting through Toll-like receptor 3 (TLR3). Alexopoulou L et al. (2001) Nature 413:732-8. Further, it has also been recently reported that certain single-stranded RNAs, generally including guanosine (G) and uridine (U), and particularly including certain sequence motifs, are also immunostimulatory. Lipford et al. US 2003/0232074 A1.

In efforts to develop siRNA for clinical application, it has recently become apparent that at least some siRNA are also immunostimulatory. In some instances it may be desirable to have both gene silencing and immunostimulation. However, in other settings it may instead be desirable to have gene silencing without accompanying immunostimulation.

### SUMMARY OF THE INVENTION

The invention provides compositions and methods relating to siRNA characterized by certain nucleotide modifications within the sense strand, such that the resulting siRNA with modification is less immunostimulatory than the corresponding siRNA without modification. The modification in the sense strand has little or no effect on the ability of the siRNA to silence target genes.

In one aspect the invention is a composition including a double-stranded short interfering ribonucleic acid (siRNA) having a sense strand and an antisense strand, each strand having a 5′ end and a 3′ end, wherein the antisense strand is complementary to a target sequence and wherein the sense strand comprises at least one modified nucleotide having a sugar with a 2′ modification, with proviso that the modified nucleotide having the sugar with the 2′ modification is not a locked nucleic acid (LNA) or a 2′-O-methyl nucleotide.

In one aspect the invention is a method for reducing immunostimulatory potential of a double-stranded short interfering ribonucleic acid (siRNA), said siRNA having a sense strand and an antisense strand, each strand having a 5′ end and a 3′ end, wherein the antisense strand is complementary to a target sequence. The method includes the step of introducing into the sense strand of the siRNA at least one modified nucleotide having a sugar with a 2′ modification, with proviso that the modified nucleotide having the sugar with the 2′ modification is not a locked nucleic acid (LNA) or a 2′-O-methyl nucleotide.

In one aspect the invention is a method for reducing expression of a gene having a target sequence. The method according to this aspect includes the step of contacting a cell comprising the gene having the target sequence with an effective amount of a double-stranded short interfering ribonucleic acid (siRNA) having a sense strand and an antisense strand, each strand having a 5′ end and a 3′ end, wherein the antisense strand is complementary to the target sequence and wherein the sense strand comprises at least one modified nucleotide having a sugar with a 2′ modification, with proviso that the modified nucleotide having the sugar with the 2′ modification is not a locked nucleic acid (LNA) or a 2′-O-methyl nucleotide, to reduce expression of the gene having the target sequence.

In one embodiment the sense strand including the modified nucleotide having the sugar with the 2′ modification is a sense strand including only one modified nucleotide having a sugar with a 2′ modification.

In one embodirnent the sense strand including the modified nucleotide having the sugar with the 2′ modification is a sense strand including a plurality of modified nucleotides having a sugar with a 2′ modification, wherein each modified nucleotide having the sugar with the 2′ modification is selected independently of any other.

In one embodiment the 2′ modification is selected from the group consisting of 2′-O-alkyl, 2′-O-alkenyl, and 2′-O-alkinyl, with proviso that 2′-O-alkyl excludes 2′-O-methyl.

In one embodiment the 2′ modification is selected from the group consisting of 2′-methoxyethyl, 2′-O-allyl, 2′-propinyl, 2′-aminopropargyl, 2′-O-(3-aminopropyl), 2′-O-propyl, and 2′-O-butyl.

In one embodiment the 2′ modification is selected from the group consisting of 2′-deoxy, 2′-fluoro, and 2′-amino.

In one embodiment the 2′ modification is 2′-fluoro.

In one embodiment the 2′ modification is selected from 2′-O-alkenyl, 2′-O-alkinyl, 2′-methoxyethyl, 2′-aminopropargyl, 2′-O-(3-aminopropyl), and 2′-amino.

In one embodiment the at least one modified nucleotide having the sugar with the 2′ modification occurs at the 5′ end of the sense strand. In one embodiment the at least one modified nucleotide having the sugar with the 2′ modification occurs at the 5′ end of the sense strand, exclusive of any overhang.

In one embodiment the at least one modified nucleotide having the sugar with the 2′ modification occurs at the 3′ end of the sense strand. In one embodiment the at least one modified nucleotide having the sugar with the 2′ modification occurs at the 3′ end of the sense strand, exclusive of any overhang.

In one embodiment the at least one modified nucleotide having the sugar with the 2′ modification occurs internal with respect to the 5′ end and the 3′ end of the sense strand. In one embodiment the at least one modified nucleotide having the sugar with the 2′ modification occurs internal with respect to the 5′ end and the 3′ end of the sense strand, exclusive of any overhang.

In one embodiment the sense strand includes at least one modified nucleotide having the sugar with the 2′ modification at the 5′ end of the sense strand and at least one modified nucleotide having the sugar with the 2′ modification at the 3′ end of the sense strand. In one embodiment the sense strand includes at least one modified nucleotide having the sugar with the 2′ modification at the 5′ end of the sense strand and at least one modified nucleotide having the sugar with the 2′ modification at the 3′ end of the sense strand, exclusive of any overhang.

In one embodiment the sense strand has a phosphodiester backbone.

In one embodiment the sense strand has a stabilized backbone including at least one stabilized internucleotide linkage.

In one embodiment the sense strand has a stabilized backbone including at least one stabilized internucleotide linkage selected from the group consisting of thioformacetal, phosphorothioate, methylphosphonate, boranophosphonate, and formacetate.

### BRIEF DESCRIPTION OF THE DRAWINGS

FIG. 1 is a group of four graphs depicting cytokine production by human peripheral blood mononuclear cells (PBMC). Indicated concentrations of indicated double-stranded siRNA (sense (s):antisense (as)) in the presence of DOTAP were incubated with human PBMC and amount of IFN-alpha (pg/ml; panels A and C) or IL-12p40 (pg/ml; panels B and D) was determined in the supernatant 24 h later by ELISA. RNA sequences for panels A and B are as follows: MAPK2 s, SEQ ID NO: 1; MAPK2 as, SEQ ID NO:2; MAPK2 Exp27 s, SEQ ID NO:3; MAPK2 Exp27 as, SEQ ID NO:4; MAPK2 Exp30 s, SEQ ID NO:3; MAPK2 Exp30 as, SEQ ID NO:5. RNA sequences for panels C and D are as follows: Lamin AC s, SEQ ID NO:6; Lamin AC as, SEQ ID NO:7; Lamin AC Exp27 s, SEQ ID NO:8; Lamin AC Exp27 as, SEQ ID NO:9; Lamin AC Exp30 s, SEQ ID NO:8; Lamin AC Exp30 as, SEQ ID NO:10.

FIG. 2 is a group of twelve graphs depicting cytokine production by human PBMC. Indicated concentrations of indicated species of RNA (double-stranded siRNA (sense (s):antisense (as)); sense strand alone (s); and antisense strand alone (as)) in the presence of DOTAP were incubated with human PBMC and amount of IFN-alpha (pg/ml; panels A and C) or IL-12p40 (pg/ml; panels B and D) was determined in the supernatant 24 h later by ELISA. RNA sequences for panels A and B are as follows: MAPK2 s, SEQ ID NO:1; MAPK2 as, SEQ ID NO:2; MAPK2 Exp27 s, SEQ ID NO:3; MAPK2 Exp27 as, SEQ ID NO:4; MAPK2 Exp30s, SEQ ID NO:3; MAPK2 Exp30 as, SEQ ID NO:5. RNA sequences for panels C and D are as follows: Lamin AC s, SEQ ID NO:6; Lamin AC as, SEQ ID NO:7; Lamin AC Exp27 s, SEQ ID NO:8; Lamin AC Exp27 as, SEQ ID NO:9; Lamin AC Exp30 s, SEQ ID NO:8; Lamin AC Exp30 as, SEQ ID NO:10.

### DETAILED DESCRIPTION OF THE INVENTION

RNA interference, including short interfering RNA (siRNA) technology, has become an important tool for down-regulation of specific genes, and siRNA therapeutics are already in development. Synthetic siRNA generally consists of double-stranded oligoribonucleotides 21-23 nucleotides in length with phosphodiester backbone. However, beside the specific gene-targeting effect of siRNA, unspecific effects of this technology have been described recently. siRNA has been shown to induce unspecific activation of the innate immune system, including up-regulation of certain cytokines, e.g. type I and/or type II interferon as well as IL-12, IL-6 and/or TNF-alpha production. The origin of these effects is thought to be activation of Toll-like receptors like TLR7, TLR8 and/or TLR3 by siRNA.

While activation of the immune system is often a desired effect, in the context of RNA silencing the unspecific activation of the immune system might interfere with the actual mode of action of siRNA and can significantly alter the outcome of treatment.

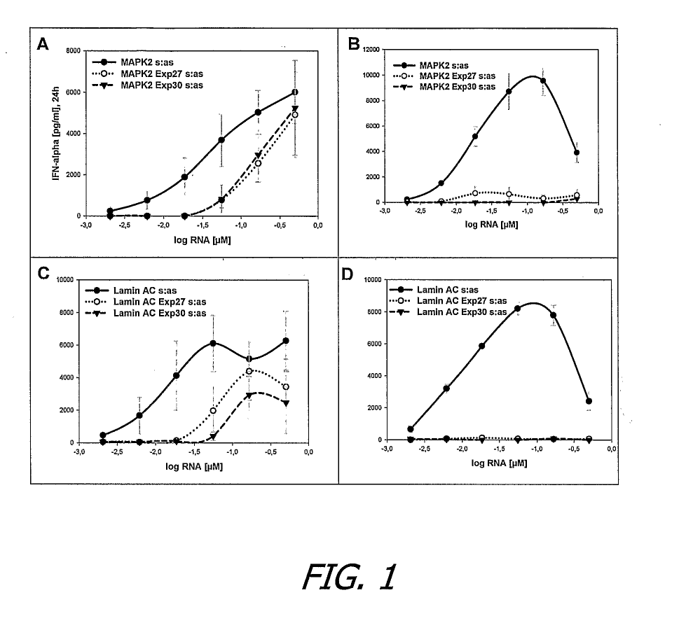

In examples described below, the immunostimulatory activity of certain siRNA constructs, characterized by certain modifications of 2′ nucleotide sugars in specific locations, were surprisingly found to have reduced immunostimulatory properties without significant compromise of their gene silencing properties. siRNA derived from the sequences of the MAPK2 (Erk2) and Lamin AC genes (Table 1) induced significant cytokine production when incubated with human PBMC in the presence of the cationic lipid N-[1-(2,3-dioleoyloxy)propyl]-N,N,N-trimethyl ammonium (DOTAP; FIG. 1). Induction of cytokines is thought to be induced by immunostimulatory sequences present in the antisense and/or sense strand of the siRNAs.

It was discovered according to the invention that chemical modifications within the sense and antisense strand can significantly suppress the immunostimulatory activity of the siRNAs. Introduction of 2′ sugar modifications at the 5′ and 3′ end of the sense strand (FIG. 1) and additional 2′ sugar modifications at the 3′ end of the antisense strand completely abolished IL-12p40 and TNF-alpha production of the siRNAs and significantly reduced IFN-alpha production.

Surprisingly, it was discovered according to the invention that the introduced modifications affected only the immunostimulatory activity of the sense strand (s, single-stranded RNA) and the double-stranded siRNA containing the sense and antisense strand, but not the antisense strand (as, single-stranded RNA) that still retained most of its immunostimulatory activity. Therefore, a suppression of the stimulatory activity of a dsRNA or siRNA appears to be possible by modifying the sense and not the antisense strand. This is of importance as it is thought that only the antisense strand is responsible for the siRNA silencing effect, so that chemical modifications to control immunostimulation can be introduced into the sense strand without affecting the antisense strand and, therefore, without affecting the silencing effect.

It was also surprisingly discovered according to the invention that modifications of the RNA sense strand only introduced at the very 5′ and 3′ ends, and, particularly, not in a potential immunostimulatory sequence, still led to strong or complete suppression of inimunostimulatory activity.

In addition, it was surprisingly discovered according to the invention that even a single 2′ modification in an immunostimulatory single strand affects the immune response strongly, indicating that a single such modification in the sense strand can be sufficient to influence immunostimulatory activity of the siRNA or dsRNA.

In a recent publication from Hornung et al. (Nature Medicine 11:263-70, 2005), it was reported that locked nucleic acid (LNA) modifications of an immunostimulatory motif at the 3′ end diminished the immunostimulatory properties of siRNA. In contrast to the report by Homung et al., it was discovered according to the invention that, unexpectedly, the modifications do not need to be within an immunostimulatory motif and modification of the sense strand alone to be non-stimulatory is sufficient to suppress immunostimulatory activity.

The invention in one aspect relates generally to compositions and methods involving double-stranded siRNA that include certain modifications. The specific modifications reduce the immunostimulatory potential of the siRNA compared to corresponding siRNA without the modifications. As used herein, siRNA shall refer to a particular type of isolated double-stranded ribonucleic acid (RNA) molecule characterized by a length of about 21-23 nucleotides, a single-stranded sense (s) strand and a single-stranded antisense (as) strand, wherein the antisense strand has a nucleotide sequence complementary to a target nucleotide sequence, which RNA molecule, when delivered into a cell expressing a protein encoded by the target sequence, reduces the amount of target nucleotide sequence (and the encoded protein) in the cell.

The sense and antisense strands of siRNA have nucleotide sequences which are strictly or at least substantially complementary to each other, such that they can form a stable duplex structure under suitable conditions, in vivo or in vitro. In certain embodiments one or both ends of either strand can extend beyond the corresponding end or ends of the other strand in the duplex structure, thereby allowing short overhanging sequence (generally 1-2 nucleotides long) at either or both ends of the siRNA.

The siRNA will generally include nucleotide subunits having canonical nucleobases common to RNA, e.g., adenine, cytosine, guanine, and uracil, but is not so limited. Other nucleobases, including but not limited to thymine and hypoxanthine, can also be present in some embodiments.

As used herein in reference to any RNA molecule, immunostimulatory potential refers to the capacity of the RNA molecule to stimulate an immune response, e.g., to stimulate a cell of the immune system to become activated to proliferate, differentiate, increase expression of secreted products associated with immune cell activation, increase expression of cell surface markers or co-stimulatory molecules associated with immune cell activation, or any combination thereof. Secreted products associated with immune cell activation are well known in the art and can include, without limitation, cytokines, chemokines, and antibodies.

As a feature of the invention, the sense strand of siRNA of the invention includes a modified nucleotide having a sugar with a 2′ modification, with the proviso that the modified nucleotide having the sugar with the 2′ modification is not a locked nucleic acid (LNA) or a 2′-O-methyl nucleotide. The sense strand can include only a single modified nucleotide having a sugar with a 2′ modification, or it can contain two or more modified nucleotide having a sugar with a 2′ modification, each selected independently of any other. In one embodiment the sense strand includes only modified nucleotides having a sugar with a 2′ modification, each selected independently of any other. More typically, the sense strand will include one to six modified nucleotides having a sugar with a 2′ modification, each selected independently of any other. When there is more than a single modified nucleotide having a sugar with a 2′ modification, the modified nucleotides having a sugar with a 2′ modification can occur, as their number permits, as adjacent nucleotides, as non-adjacent nucleotides, or as a combination of adjacent and non-adjacent nucleotides.

As used herein, a nucleotide refers to a sugar (e.g., ribose or deoxyribose) linked to a phosphate group and to an exchangeable organic base (e.g., a nucleobase), which is either a substituted pyrimidine (e.g., cytosine, thymine, or uracil) or substituted purine (e.g., adenine or guanine). As used herein, nucleotides having cytosine, thymine, uracil, adenine, or guanine as their nucleobase are denoted by their conventional single letter symbols C, T, U, A, or G, respectively. Ribonucleotides include canonical C, U, A, and G ribonucleotides, but are not so limited.

As used herein in reference to any species of RNA, a modified nucleotide having a sugar with a 2′ modification refers to a nucleotide in which the sugar has a substituent at the 2′ position that is non-standard for a ribonucleotide. In one embodiment the sugar with the 2′ modification is a 2′ deoxyribose sugar, such that the corresponding nucleotide is a deoxyribonucleotide. In one embodiment the 2′ modification is selected from the group consisting of 2′-O-alkyl, 2′-O-alkenyl, and 2′-O-alkinyl, with proviso that 2′-O-alkyl excludes 2′-O-methyl. In one embodiment the 2′ modification is selected from the group consisting of 2′-methoxyethyl, 2′-O-allyl, 2′-propinyl, 2′-aminopropargyl, 2′-O-(3-aminopropyl), 2′-O-propyl, and 2′-O-butyl. In one embodiment the 2′ modification is selected from the group consisting of 2′-deoxy, 2′-fluoro-2′-deoxy (i.e., 2′-fluoro), and 2′-amino-2′-deoxy (i.e., 2′-amino). In one embodiment the 2′ modification is 2′-fluoro. In one embodiment the 2′ modification is selected from 2′-O-alkenyl, 2′-O-alkinyl, 2′-methoxyethyl, 2′-aminopropargyl, 2′-O-(3-aminopropyl), and 2′-amino.

As used herein, a locked nucleic acid (LNA) refers to an RNA derivative in which the ribose ring is constrained by a methylene linkage between the 2′-oxygen and the 4′-carbon. Wahlestedt C et al. (2000) Proc Natl Acad Sci USA 97:5633-8.

Generally speaking, a modified nucleotide having a sugar with a 2′ modification can occur anywhere along the sense strand. In particular, in one embodiment a modified nucleotide having a sugar with a 2′ modification occurs at the 5′ end of the sense strand. In one embodiment a modified nucleotide having a sugar with a 2′ modification occurs at the 3′ end of the sense strand. In one embodiment a modified nucleotide having a sugar with a 2′ modification occurs at the 5′ end of the sense strand and at the 3′ end of the sense strand. A modified nucleotide having a sugar with a 2′ modification need not occur at an end of the sense strand, but rather can occur between the ends of the sense strand, i.e., internal with respect to the 5′ end and the 3′ end of the sense strand. In certain embodiments a modified nucleotide having a sugar with a 2′ modification occurs at one or both of the 5′ end and the 3′ end of the sense strand, and also internal with respect to the 5′ end and the 3′ end of the sense strand.

The nucleotide sequence of the sense strand, the antisense strand, or both the sense strand and the antisense strand can optionally include an immunostimulatory sequence or motif. In one embodiment the immunostimulatory sequence or motif is 5′-RURGY-3′, wherein each R independently represents purine ribonucleotide and Y represents pyrimidine ribonucleotide. In various embodiments 5′-RURGY-3′ specifically can include but is not limited to 5′-GUGGU-3′,5′-GUGGC-3′,5′-GUAGU-3′, 5′-GUAGC-3′,5′-AUGGU-3′,5′-AUGGC-3′,5′-AUAGU-3′, and 5′-AUAGC-3′. In one embodiment the immunostimulatory sequence or motif is 5′-GUAGUGU-3′. In one embodiment the immunostimulatory sequence or motif is 5′-GUUGB-3′, wherein B represents U, G, or C. In various embodiments 5′-GUUGB-3′ specifically includes 5′-GUUGU-3′,5′-GUUGG-3′, and 5′-GUUGC-3′. In one embodiment the immunostimulatory sequence or motif is 5′-GUGUG-3′. In one embodiment the immunostimulatory sequence or motif is 5′-GUGUUUAC-3′. In one embodiment the immunostimulatory sequence or motif is 5′-GUAGGCAC-3′. In one embodiment the immunostimulatory sequence or motif is 5′-CUAGGCAC-3′. In one embodiment the immunostimulatory sequence or motif is 5′-CUCGGCAC-3′.

When the sense strand includes an identifiable immunostimulatory sequence or motif, the modified nucleotide having a sugar with a 2′ modification in one embodiment occurs within the identifiable immunostimulatory sequence or motif.

Alternatively, and significantly, when the sense strand includes an identifiable immunostimulatory sequence or motif, the modified nucleotide having a sugar with a 2′ modification in one embodiment occurs outside of the identifiable immunostimulatory sequence or motif. When the sense strand includes an identifiable immunostimulatory sequence or motif and the modified nucleotide having a sugar with a 2′ modification occurs outside of the identifiable immunostimulatory sequence or motif, in one embodiment the modified nucleotide having a sugar with a 2′ modification occurs immediately adjacent to the identifiable immunostimulatory sequence or motif. Immediately adjacent to, in one embodiment, is immediately 5′ with respect to the immunostimulatory sequence or motif. Immediately adjacent to, in one embodiment, is immediately 3′ with respect to the immunostimulatory sequence or motif. In other embodiments in which the sense strand includes an identifiable immunostimulatory sequence or motif and the modified nucleotide having a sugar with a 2′ modification occurs outside of the identifiable immunostimulatory sequence or motif, the modified nucleotide having a sugar with a 2′ modification occurs at least one nucleotide removed from the immunostimulatory sequence or motif. The number of nucleotides between the modified nucleotide having a sugar with a 2′ modification and the immunostimulatory sequence or motif can be, in various embodiments, 1, 2, 3, 4, 5, 6, 7, 8, 9, 10, 11, 12, 13, 14, 15, 16, 17, or 18.

Any nucleotide of the sense strand, including any modified nucleotide having a sugar with a 2′ modification, as defined above, can optionally include a modification involving the phosphate group. In one embodiment the sense strand has a phosphodiester backbone, i.e., the nucleotides of the sense strand are linked one to the next by phosphodiester linkages. Such phosphodiester linkages and phosphodiester backbone are typical of nucleic acid molecules as they occur in nature, and they are relatively susceptible to nuclease cleavage in vivo.

In one embodiment the sense strand has a stabilized backbone. The stabilized backbone includes at least one stabilized internucleotide linkage, resulting in a backbone that is relatively resistant to nuclease cleavage in vivo or in vitro compared to phosphodiester backbone. In one embodiment the stabilized backbone includes only stabilized internucleotide linkages. In one embodiment the stabilized internucleotide linkage is selected from the group consisting of thioformacetal, phosphorothioate, methylphosphonate, boranophosphonate, and formacetate. In one embodiment the stabilized internucleotide linkage is a phosphorothioate linkage.

The siRNA of the invention can be synthesized using automated techniques and devices employing, for example, either phosphoramidate or H-phosphonate chemistries. Methods for making other nucleic acid backbone modifications and substitutions have been described and are contemplated for use in the invention. Uhlmann E et al. (1990) Chem Rev 90:544; Goodchild J (1990) Bioconjugate Chem 1:165.

The sense and antisense strands can be synthesized separately. Alternatively, the sense and antisense strands can be synthesized as a single construct and then treated to clip off or otherwise remove intervening or extraneous nucleotides or linking moieties. No matter how they are synthesized, the desired siRNA or component sense and antisense strands are preferably isolated from extraneous synthesis reagents and, optionally, purified prior to use.

The compositions of the invention are believed to be useful in any situation calling for the use of siRNA. Thus the target sequence can be any suitable target sequence. Clinical situations calling for the use of siRNA include, without limitation, treatment of subjects having cancer, treatment of subjects having infectious disease, treatment of subjects having autoimmune disease, treatment of subjects having transplant rejection, and treatment of subjects having allergy or asthma. Those skilled in the art will be familiar with how to select a suitable target sequence and assess the efficacy of the RNA interference for that target. Methods for assess efficacy of the RNA interference for a particular target can be accomplished using standard techniques of nucleotide and protein analysis, such as quantitative reverse transcriptase-polymerase chain reaction (qRT-PCR), immunoblotting, and enzyme-linked immunosorbent assay (ELISA), provided such techniques are suitably adapted to the particular target, for example through proper selection of amplification primers and antibodies.

The invention in one aspect provides a method for reducing immunostimulatory potential of an siRNA. This method in one embodiment can be used to reduce the immunostimulatory potential of a previously characterized siRNA. In one embodiment the method can be used to reduce the immunostimulatory potential of a previously uncharacterized siRNA, for example in designing and synthesizing an siRNA for the first time. The method includes the step of introducing a modified nucleotide having a sugar with a 2′ modification into a sense strand of a double-stranded siRNA having a sense strand and an antisense strand, wherein the antisense strand is complementary to a target sequence, with proviso that the modified nucleotide having the sugar with the 2′ modification is not a locked nucleic acid (LNA) or a 2′-O-methyl nucleotide. As used herein with reference to this aspect of the invention, introducing a modified nucleotide having a sugar with a 2′ modification refers to substituting a modified nucleotide having a sugar with a 2′ modification, as defined above, in the place of an existing or naturally occurring nucleotide. For example, where in an existing siRNA the antisense strand has a G that calls for a C on the sense strand, a deoxycytidine (dC) is substituted in place of the C. The step of introducing a modified nucleotide having a sugar with a 2′ modification into a sense strand thus typically involves designing and performing the synthesis of the sense strand in such manner that the desired modified nucleotide is incorporated into the product sense strand in the desired location.

The invention in one aspect is a method of practicing RNA interference using an siRNA of the invention. More particularly, the method according to this aspect of the invention is a method for reducing expression of a gene having a target sequence. As used herein, in one embodiment reducing expression of a gene having a target sequence refers to reducing the amount of messenger RNA transcribed from a particular gene of interest. Also as used herein, in one embodiment reducing expression of a gene having a target sequence refers to reducing the amount of protein product present in a cell encoded by a particular gene of interest. The method according to this aspect of the invention includes the step of contacting a cell including the gene having the target sequence with an effective amount of a double-stranded short interfering ribonucleic acid (siRNA) having a sense strand and an antisense strand, wherein the antisense strand is complementary to the target sequence and wherein the sense strand includes a modified nucleotide having a sugar with a 2′ modification, with proviso that the modified nucleotide having the sugar with the 2′ modification is not a locked nucleic acid (LNA) or a 2′-O-methyl nucleotide, to reduce expression of the gene having the target sequence. The method according to this aspect of the invention can be performed in vitro and in vivo. When practicing the method in vivo, the contacting step further entails administering a composition of the invention to a subject.

siRNA of the invention may be of particular use in the treatment of subjects having a cancer, subjects having an infectious disease, subjects having an autoimmune disease, subjects having allergy, and subjects having asthma, but it is not so limited.

“Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

As used herein, a subject having a cancer refers to a subject that has detectable cancerous cells.

A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas.

An “infectious disease” as used herein, refers to a disorder arising from the invasion of a host, superficially, locally, or systemically, by an infectious microorganism. Infectious microorganisms include bacteria, viruses, parasites and fungi.

As used herein, a subject having an infectious disease refers to a subject that has been exposed to an infectious organism and has acute or chronic detectable levels of the organism in the body. Exposure to the infectious organism generally occurs with the external surface of the subject, e.g., skin or mucosal membranes and/or refers to the penetration of the external surface of the subject by the infectious organism.

Examples of viruses that have been found in humans include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Both gram negative and gram positive bacteria serve as antigens in vertebrate animals. Such gram positive bacteria include, but are not limited to, Pasteurella species, Staphylococci species, and Streptococcus species. Gram negative bacteria include, but are not limited to, Escherichia coli, Pseudomonas species, and Salmonella species. Specific examples of infectious bacteria include but are not limited to, Helicobacter pyloris, Borrelia burgdorferi, Legionella pneumophilia, Mycobacteria sps (e.g. M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Examples of fungi include Cryptococcus neoformans, Histoplasma capsulatum, Coccidioides immitis, Blastomyces dermatitidis, Chlanydia trachomatis, Candida albicans.

Other infectious organisms (i.e., protists) include Plasmodium spp. such as Plasmodium falciparum, Plasmodium malariae, Plasmodium ovale, and Plasmodium vivax and Toxoplasma gondii. Blood-borne and/or tissues parasites include Plasmodium spp., Babesia microti, Babesia divergens, Leishmania tropica, Leishmania spp., Leishmania braziliensis, Leishmania donovani Trypanosoma gambiense and Trypanosoma rhodesiense (African sleeping sickness), Trypanosoma cruzi (Chagas' disease), and Toxoplasma gondii.

Other medically relevant microorganisms have been described extensively in the literature, e.g., see C. G. A Thomas, Medical Microbiology, Bailliere Tindall, Great Britain 1983, the entire contents of which is hereby incorporated by reference.

The siRNA of the invention are also useful for treating and preventing autoimmune disease. Autoimmune disease is a class of diseases in which a subject's own antibodies react with host tissue or in which immune effector T cells are autoreactive to endogenous self peptides and cause destruction of tissue. Thus an immune response is mounted against a subject's own antigens, referred to as self antigens. Autoimmune diseases include but are not limited to rheumatoid arthritis, Crohn's disease, multiple sclerosis, systemic lupus erythematosus (SLE), autoimmune encephalomyelitis, myasthenia gravis (MG), Hashimoto's thyroiditis, Goodpasture's syndrome, pemphigus (e.g., pemphigus vulgaris), Grave's disease, autoimmune hemolytic anemia, autoimmune thrombocytopenic purpura, scleroderma with anti-collagen antibodies, mixed connective tissue disease, polymyositis, pernicious anemia, idiopathic Addison's disease, autoimmune-associated infertility, glomerulonephritis (e.g., crescentic glomerulonephritis, proliferative glomerulonephritis), bullous pemphigoid, Sjögren's syndrome, insulin resistance, and autoimmune diabetes mellitus.

As used herein, an allergy refers to acquired hypersensitivity to a substance (allergen). Allergic conditions include but are not limited to eczema, allergic rhinitis or coryza, hay fever, allergic conjunctivitis, bronchial asthma, urticaria (hives) and food allergies, other atopic conditions including atopic dermatitis; anaphylaxis; drug allergy; and angioedema. Allergic diseases include but are not limited to rhinitis (hay fever), asthma, urticaria, and atopic dermatitis.

As used herein, a subject having an allergy is a subject that has an allergic reaction in response to an allergen.

An allergen refers to a substance (antigen) that can induce an allergic or asthmatic response in a susceptible subject. The list of allergens is enormous and can include pollens, insect venoms, animal dander dust, fungal spores and drugs (e.g. penicillin). Examples of natural, animal and plant allergens include but are not limited to proteins specific to the following genuses: Canis (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poapratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

As used herein, asthma refers to a disorder of the respiratory system characterized by inflammation, narrowing of the airways, and increased reactivity of the airways to inhaled agents. Asthma is frequently, although not exclusively, associated with an atopic or allergic condition. Symptoms of asthma include recurrent episodes of wheezing, breathlessness, and chest tightness, and coughing, resulting from airflow obstruction.

Airway inflammation associated with asthma can be detected through observation of a number of physiological changes, such as, denudation of airway epithelium, collagen deposition beneath basement membrane, edema, mast cell activation, inflammatory cell infiltration, including neutrophils, inosineophils, and lymphocytes. As a result of the airway inflammation, asthma patients often experience airway hyper-responsiveness, airflow limitation, respiratory symptoms, and disease chronicity. Airflow limitations include acute bronchoconstriction, airway edema, mucous plug formation, and airway remodeling, features which often lead to bronchial obstruction. In some cases of asthma, sub-basement membrane fibrosis may occur, leading to persistent abnormalities in lung function.

As used herein, a subject having asthma is a subject that has a disorder of the respiratory system characterized by inflammation, narrowing of the airways and increased reactivity of the airways to inhaled agents. Asthma is frequently, although not exclusively, associated with atopic or allergic symptoms. Asthma is also frequently, although not exclusively, associated with contact with an initiator. An “initiator” as used herein refers to a composition or environmental condition which triggers asthma Initiators include, but are not limited to, allergens, cold temperatures, exercise, viral infections, SO₂.

siRNA of the invention can be used either alone or combined with other therapeutic agents. The other therapeutic agent in one embodiment is another siRNA of the invention. The siRNA and other therapeutic agent may be administered simultaneously or sequentially. When the other therapeutic agents are administered simultaneously, they can be administered in the same or separate formulations, but are administered at the same time. The other therapeutic agents are administered sequentially with one another and with siRNA, when the administration of the other therapeutic agents and the siRNA is temporally separated. The separation in time between the administration of these compounds may be a matter of minutes or it may be longer. Other therapeutic agents include but are not limited to anti-microbial agents, anti-cancer agents, anti-allergy agents, etc.

The siRNA of the invention may be administered to a subject with an anti-microbial agent. An anti-microbial agent, as used herein, refers to a naturally-occurring or synthetic compound which is capable of killing or inhibiting infectious microorganisms. The type of anti-microbial agent useful according to the invention will depend upon the type of microorganism with which the subject is infected or at risk of becoming infected. Anti-microbial agents include but are not limited to anti-bacterial agents, anti-viral agents, anti-fungal agents and anti-parasitic agents. Phrases such as “anti-infective agent”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Antibiotics are low molecular weight molecules which are produced as secondary metabolites by cells, such as microorganisms. In general, antibiotics interfere with one or more bacterial functions or structures which are specific for the microorganism and which are not present in host cells. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites.

Examples of anti-parasitic agents, also referred to as parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, rifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide some of which are used alone or in combination with others.

Antibacterial agents kill or inhibit the growth or function of bacteria. A large class of antibacterial agents is antibiotics. Antibiotics, which are effective for killing or inhibiting a wide range of bacteria, are referred to as broad spectrum antibiotics. Other types of antibiotics are predominantly effective against the bacteria of the class gram-positive or gram-negative. These types of antibiotics are referred to as narrow spectrum antibiotics. Other antibiotics which are effective against a single organism or disease and not against other types of bacteria, are referred to as limited spectrum antibiotics.

Antibacterial agents are sometimes classified based on their primary mode of action. In general, antibacterial agents are cell wall synthesis inhibitors, cell membrane inhibitors, protein synthesis inhibitors, nucleic acid synthesis or functional inhibitors, and competitive inhibitors.

Antiviral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleotide analogues), maturation of new virus proteins (e.g. protease inhibitors), and budding and release of the virus.

Nucleotide analogues are synthetic compounds which are similar to nucleotides, but which have an incomplete or abnormal deoxyribose or ribose group. Once the nucleotide analogues are in the cell, they are phosphorylated, producing the triphosphate formed which competes with normal nucleotides for incorporation into the viral DNA or RNA. Once the triphosphate form of the nucleotide analogue is incorporated into the growing nucleic acid chain, it causes irreversible association with the viral polymerase and thus chain termination. Nucleotide analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, zidovudine (azidothymidine), imiquimod, and resimiquimod.

The interferons are cytokines which are secreted by virus-infected cells as well as immune cells. The interferons function by binding to specific receptors on cells adjacent to the infected cells, causing the change in the cell which protects it from infection by the virus. α and β-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection. At the dosages which are effective for anti-viral therapy, interferons have severe side effects such as fever, malaise and weight loss.

Anti-viral agents useful in the invention include but are not limited to immunoglobulins, amantadine, interferons, nucleotide analogues, and protease inhibitors. Specific examples of anti-virals include but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, immidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g. chitinase) or immunosuppression (501 cream).

The siRNA of the invention may also be administered in conjunction with an anti-cancer therapy. Anti-cancer therapies include cancer medicaments, radiation and surgical procedures. As used herein, a “cancer medicament” refers to an agent which is administered to a subject for the purpose of treating a cancer. As used herein, “treating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Various types of medicaments for the treatment of cancer are described herein. For the purpose of this specification, cancer medicaments are classified as chemotherapeutic agents, immunotherapeutic agents, cancer vaccines, hormone therapy, and biological response modifiers.

The chemotherapeutic agent may be selected from the group consisting of methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS farnesyl transferase inhibitor, farnesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH4556, CS-682, 9-AC, AG3340, AG3433, Incel/VX-710, VX-853, ZD0101, ISI641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodalemozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT (Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP 16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin-2, Mitoguazone (methyl-GAG; methyl glyoxal bis-guanylhydrazone; MGBG), Pentostatin (2′ deoxycoformycin), Semustine (methyl-CCNU), Teniposide (VM-26) and Vindesine sulfate, but it is not so limited.

The immunotherapeutic agent may be selected from the group consisting of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab and ImmuRAIT-CEA, but it is not so limited.

The cancer vaccine may be selected from the group consisting of EGF, Anti-idiotypic cancer vaccines, Gp75 antigen, GMK melanoma vaccine, MGV ganglioside conjugate vaccine, Her2/neu, Ovarex, M-Vax, O-Vax, L-Vax, STn-KHL theratope, BLP25 (MUC-1), liposomal idiotypic vaccine, Melacine, peptide antigen vaccines, toxin/antigen vaccines, MVA-based vaccine, PACIS, BCG vacine, TA-HPV, TA-CIN, DISC-virus and ImmuCyst/TheraCys, but it is not so limited.

The siRNA of the invention may be administered to a subject with an asthma/allergy medicament. An “asthma/allergy medicament” as used herein is a composition of matter which reduces the symptoms of, prevents the development of, or inhibits an asthmatic or allergic reaction. Various types of medicaments for the treatment of asthma and allergy are described in the Guidelines For The Diagnosis and Management of Asthma, Expert Panel Report 2, NIH Publication No. 97/4051, Jul. 19, 1997, the entire contents of which are incorporated herein by reference. The summary of the medicaments as described in the NIH publication is presented below. In most embodiments the asthma/allergy medicament is useful to some degree for treating both asthma and allergy.

Medications for the treatment of asthma are generally separated into two categories, quick-relief medications and long-term control medications. Asthma patients take the long-term control medications on a daily basis to achieve and maintain control of persistent asthma. Long-term control medications include anti-inflammatory agents such as corticosteroids, chromolyn sodium and nedocromil; long-acting bronchodilators, such as long-acting β₂-agonists and methylxanthines; and leukotriene modifiers. The quick-relief medications include short-acting β₂ agonists, anti-cholinergics, and systemic corticosteroids. There are many side effects associated with each of these drugs and none of the drugs alone or in combination is capable of preventing or completely treating asthma.

Asthma medicaments include, but are not limited, PDE-4 inhibitors, bronchodilator/beta-2 agonists, K+channel openers, VLA-4 antagonists, neurokin antagonists, thromboxane A2 (TXA2) synthesis inhibitors, xanthines, arachidonic acid antagonists, 5 lipoxygenase inhibitors, TXA2 receptor antagonists, TXA2 antagonists, inhibitor of 5-lipox activation proteins, and protease inhibitors.

Bronchodilator/P₂ agonists are a class of compounds which cause bronchodilation or smooth muscle relaxation. Bronchodilator/β₂ agonists include, but are not limited to, salmeterol, salbutamol, albuterol, terbutaline, D2522/formoterol, fenoterol, bitolterol, pirbuerol methylxanthines and orciprenaline. Long-acting β₂ agonists and bronchodilators are compounds which are used for long-term prevention of symptoms in addition to the anti-inflammatory therapies. Long-acting β₂ agonists include, but are not limited to, salmeterol and albuterol. These compounds are usually used in combination with corticosteroids and generally are not used without any inflammatory therapy. They have been associated with side effects such as tachycardia, skeletal muscle tremor, hypokalemia, and prolongation of QTc interval in overdose.

Methylxanthines, including for instance theophylline, have been used for long-term control and prevention of symptoms. These compounds cause bronchodilation resulting from phosphodiesterase inhibition and likely adenosine antagonism. Dose-related acute toxicities are a particular problem with these types of compounds. As a result, routine serum concentration must be monitored in order to account for the toxicity and narrow therapeutic range arising from individual differences in metabolic clearance. Side effects include tachycardia, tachyarrhythmias, nausea and vomiting, central nervous system stimulation, headache, seizures, hematemesis, hyperglycemia and hypokalemia. Short-acting β₂ agonists include, but are not limited to, albuterol, bitolterol, pirbuterol, and terbutaline. Some of the adverse effects associated with the administration of short-acting β₂ agonists include tachycardia, skeletal muscle tremor, hypokalemia, increased lactic acid, headache, and hyperglycemia.

Conventional methods for treating or preventing allergy have involved the use of anti-histamines or desensitization therapies. Anti-histamines and other drugs which block the effects of chemical mediators of the allergic reaction help to regulate the severity of the allergic symptoms but do not prevent the allergic reaction and have no effect on subsequent allergic responses. Desensitization therapies are performed by giving small doses of an allergen, usually by injection under the skin, in order to induce an IgG-type response against the allergen. The presence of IgG antibody helps to neutralize the production of mediators resulting from the induction of IgE antibodies, it is believed. Initially, the subject is treated with a very low dose of the allergen to avoid inducing a severe reaction and the dose is slowly increased. This type of therapy is dangerous because the subject is actually administered the compounds which cause the allergic response and severe allergic reactions can result.

Allergy medicaments include, but are not limited to, anti-histamines, steroids, and prostaglandin inducers. Anti-histamines are compounds which counteract histamine released by mast cells or basophils. These compounds are well known in the art and commonly used for the treatment of allergy. Anti-histamines include, but are not limited to, astemizole, azelastine, betatastine, buclizine, ceterizine, cetirizine analogues, CS 560, desloratadine, ebastine, epinastine, fexofenadine, HSR 609, levocabastine, loratidine, mizolastine, norastemizole, terfenadine, and tranilast.

Prostaglandin inducers are compounds which induce prostaglandin activity. Prostaglandins function by regulating smooth muscle relaxation. Prostaglandin inducers include, but are not limited to, S-5751.

The asthma/allergy medicaments also include steroids and immunomodulators. The steroids include, but are not limited to, beclomethasone, fluticasone, triamcinolone, budesonide, corticosteroids and budesonide.

Corticosteroids include, but are not limited to, beclomethasome dipropionate, budesonide, flunisolide, fluticaosone propionate, and triamcinolone acetonide. Although dexamethasone is a corticosteroid having anti-inflammatory action, it is not regularly used for the treatment of asthma/allergy in an inhaled form because it is highly absorbed and it has long-term suppressive side effects at an effective dose. Dexamethasone, however, can be used according to the invention for the treating of asthma/allergy because when administered in combination with nucleic acids of the invention it can be administered at a low dose to reduce the side effects. Some of the side effects associated with corticosteroid include cough, dysphonia, oral thrush (candidiasis), and in higher doses, systemic effects, such as adrenal suppression, osteoporosis, growth suppression, skin thinning and easy bruising. Barnes & Peterson (1993) Am Rev Respir Dis 148:S1-S26; and Kamada A K et al. (1996) Am J Respir Crit. Care Med 153:1739-48.

Systemic corticosteroids include, but are not limited to, methylprednisolone, prednisolone and prednisone. Cortosteroids are associated with reversible abnormalities in glucose metabolism, increased appetite, fluid retention, weight gain, mood alteration, hypertension, peptic ulcer, and aseptic necrosis of bone. These compounds are useful for short-term (3-10 days) prevention of the inflammatory reaction in inadequately controlled persistent asthma. They also function in a long-term prevention of symptoms in severe persistent asthma to suppress and control and actually reverse inflammation. Some side effects associated with longer term use include adrenal axis suppression, growth suppression, dermal thinning, hypertension, diabetes, Cushing's syndrome, cataracts, muscle weakness, and in rare instances, impaired immune function. It is recommended that these types of compounds be used at their lowest effective dose (guidelines for the diagnosis and management of asthma; expert panel report to; NIH Publication No. 97-4051; July 1997).

The immunomodulators include, but are not limited to, the group consisting of anti-inflammatory agents, leukotriene antagonists, IL-4 muteins, soluble IL-4 receptors, immunosuppressants (such as tolerizing peptide vaccine), anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, and downregulators of IgE.

Leukotriene modifiers are often used for long-term control and prevention of symptoms in mild persistent asthma. Leukotriene modifiers function as leukotriene receptor antagonists by selectively competing for LTD-4 and LTE-4 receptors. These compounds include, but are not limited to, zafirlukast tablets and zileuton tablets. Zileuton tablets function as 5-lipoxygenase inhibitors. These drugs have been associated with the elevation of liver enzymes and some cases of reversible hepatitis and hyperbilirubinemia. Leukotrienes are biochemical mediators that are released from mast cells, inosineophils, and basophils that cause contraction of airway smooth muscle and increase vascular permeability, mucous secretions and activate inflammatory cells in the airways of patients with asthma.

Other immunomodulators include neuropeptides that have been shown to have immunomodulating properties. Functional studies have shown that substance P, for instance, can influence lymphocyte function by specific receptor-mediated mechanisms. Substance P also has been shown to modulate distinct immediate hypersensitivity responses by stimulating the generation of arachidonic acid-derived mediators from mucosal mast cells. McGillies J et al. (1987) Fed Proc 46:196-9 (1987). Substance P is a neuropeptide first identified in 1931. Von Euler and Gaddum J Physiol (London) 72:74-87 (1931). Its amino acid sequence was reported by Chang et al. in 1971. Chang M M et al. (1971) Nature New Biol 232:86-87. The immunoregulatory activity of fragments of substance P has been studied by Siemion I Z et al. (1990) Molec Immunol 27:887-890 (1990).

Another class of compounds is the down-regulators of IgE. These compounds include peptides or other molecules with the ability to bind to the IgE receptor and thereby prevent binding of antigen-specific IgE. Another type of downregulator of IgE is a monoclonal antibody directed against the IgE receptor-binding region of the human IgE molecule. Thus, one type of downregulator of IgE is an anti-IgE antibody or antibody fragment. Anti-IgE is being developed by Genentech. One of skill in the art could prepare functionally active antibody fragments of binding peptides which have the same function. Other types of IgE downregulators are polypeptides capable of blocking the binding of the IgE antibody to the Fc receptors on the cell surfaces and displacing IgE from binding sites upon which IgE is already bound.

One problem associated with downregulators of IgE is that many molecules do not have a binding strength to the receptor corresponding to the very strong interaction between the native IgE molecule and its receptor. The molecules having this strength tend to bind irreversibly to the receptor. However, such substances are relatively toxic since they can bind covalently and block other structurally similar molecules in the body. Of interest in this context is that the a chain of the IgE receptor belongs to a larger gene family where, e.g., several of the different IgG Fc receptors are contained. These receptors are absolutely essential for the defense of the body against, e.g., bacterial infections. Molecules activated for covalent binding are, furthermore, often relatively unstable and therefore they probably have to be administered several times a day and then in relatively high concentrations in order to make it possible to block completely the continuously renewing pool of IgE receptors on mast cells and basophilic leukocytes.

Chromolyn sodium and nedocromil are used as long-term control medications for preventing primarily asthma symptoms arising from exercise or allergic symptoms arising from allergens. These compounds are believed to block early and late reactions to allergens by interfering with chloride channel function. They also stabilize mast cell membranes and inhibit activation and release of mediators from inosineophils and epithelial cells. A four to six week period of administration is generally required to achieve a maximum benefit.

Anticholinergics are generally used for the relief of acute bronchospasm. These compounds are believed to function by competitive inhibition of muscarinic cholinergic receptors. Anticholinergics include, but are not limited to, ipratropium bromide. These compounds reverse only cholinerigically-mediated bronchospasm and do not modify any reaction to antigen. Side effects include drying of the mouth and respiratory secretions, increased wheezing in some individuals, and blurred vision if sprayed in the eyes.

For their use in vitro and in vivo, siRNA of the invention are generally used in an effective amount. As used herein, an effective amount refers generally to any amount that is sufficient to achieve a desired biological effect. In one embodiment an effective amount is a clinically effective amount, wherein a clinically effective amount is any amount that is sufficient to treat a subject having a disease. As used herein, treat and treating refer to reducing, eliminating, or preventing at least one sign or symptom of a disease in a subject having or at risk of having the disease. As used herein, a subject refers to a human or other mammal.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is effective to treat the particular subject. The effective amount for any particular application can vary depending on such factors as the disease or condition being treated, the particular siRNA being administered, the size of the subject, or the severity of the disease or condition. One of ordinary skill in the art can empirically determine the effective amount of a particular siRNA and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate system levels can be determined by, for example, measurement of the patient's peak or sustained plasma level of the drug. “Dose” and “dosage” are used interchangeably herein.

Generally, daily oral doses of active compounds will be from about 0.01 milligrams/kg per day to 1000 mllligrams/kg per day. It is expected that oral doses in the range of 0.5 to 50 milligrams/kg, in one or several administrations per day, will yield the desired results. Dosage may be adjusted appropriately to achieve desired drug levels, local or systemic, depending upon the mode of administration. For example, it is expected that intravenous administration would be from an order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

For any compound described herein the therapeutically effective amount can be initially determined from animal models. A therapeutically effective dose can also be determined from human data for siRNA which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

In order to promote delivery of siRNA into cells, the siRNA optionally can be presented, formulated, or otherwise combined with a cationic lipid. In one embodiment such cationic lipid is DOTAP.

For use in therapy, an effective amount of the siRNA can be administered to a subject by any mode that delivers the siRNA to the desired surface. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, and rectal.

The siRNA of the invention may be delivered to a particular tissue, cell type, or to the immune system, or both, with the aid of a vector. In its broadest sense, a “vector” is any vehicle capable of facilitating the transfer of the compositions to the target cells. The vector generally transports the siRNA, antibody, antigen, and/or disorder-specific medicament to the target cells with reduced degradation relative to the extent of degradation that would result in the absence of the vector.

In general, the vectors useful in the invention are divided into two classes: biological vectors and chemical/physical vectors. Biological vectors and chemical/physical vectors are useful in the delivery and/or uptake of therapeutic agents of the invention.

As used herein, a “chemical/physical vector” refers to a natural or synthetic molecule, other than those derived from bacteriological or viral sources, capable of delivering the siRNA and/or other medicament.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vesicles (LUVs), which range in size from 0.2-4.0 μm can encapsulate large macromolecules. RNA, DNA and intact virions can be encapsulated within the aqueous interior and be delivered to cells in a biologically active form. Fraley et al. (1981) Trends Biochem Sci 6:77.

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. In still other embodiments, the liposome may be targeted to the cancer by coupling it to a one of the immunotherapeutic antibodies discussed earlier. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTINm and LIPOFECTACE™, which are formed of cationic lipids such as N-[1-(2, 3 dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis G (1985) Trends Biotechnol 3:235-241.

In one embodiment, the vehicle is a biocompatible microparticle or implant that is suitable for implantation or administration to the mammalian recipient. Exemplary bioerodible implants that are useful in accordance with this method are described in published International Application WO 95/24929, entitled “Polymeric Gene Delivery System”. WO 95/24929 describes a biocompatible, preferably biodegradable polymeric matrix for containing an exogenous gene under the control of an appropriate promoter. The polymeric matrix can be used to achieve sustained release of the therapeutic agent in the subject.

The polymeric matrix preferably is in the form of a microparticle such as a microsphere (wherein the nucleic acid and/or the other therapeutic agent is dispersed throughout a solid polymeric matrix) or a microcapsule (wherein the nucleic acid and/or the other therapeutic agent is stored in the core of a polymeric shell). Other forms of the polymeric matrix for containing the therapeutic agent include films, coatings, gels, implants, and stents. The size and composition of the polymeric matrix device is selected to result in favorable release Idnetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid and/or the other therapeutic agent are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time. In some preferred embodiments, the nucleic acid are administered to the subject via an implant while the other therapeutic agent is administered acutely. Biocompatible microspheres that are suitable for delivery, such as oral or mucosal delivery, are disclosed in Chickering et al. (1996) Biotech Bloeng 52:96-101 and Mathiowitz E et al. (1997) Nature 386:410-414 and PCT Pat. Application WO97/03702.

Both non-biodegradable and biodegradable polymeric matrices can be used to deliver the nucleic acid and/or the other therapeutic agent to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable, particularly for the nucleic acid agents. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathalc and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The use of compaction agents may also be desirable. Compaction agents also can be used alone, or in combination with, a biological or chemical/physical vector. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake of a nucleic acid include calcium phosphate and other chemical mediators of intracellular transport, microinjection compositions, electroporation and homologous recombination compositions (e.g., for integrating a nucleic acid into a preselected location within the target cell chromosome).

The compounds may be administered alone (e.g., in saline or buffer) or using any delivery vectors known in the art. For instance the following delivery vehicles have been described: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de Haan 1995a, 1995b); live bacterial vectors (e.g., Salmonella, Escherichia coli, bacillus Calmette-Guerin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasald et al., 1998, Okada et al., 1997, Ishii et al., 1997); polymers (e.g. carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); virosomes (Gluck et al., 1992, Mengiardi et al., 1995, Cryz et al., 1998); and, virus-like particles (Jiang et al., 1999, Leibl et al., 1998).

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

The term pharmaceutically acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency.

For oral administration, the compounds (i.e., siRNA, and optionally other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA, for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the siRNA (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the siRNA (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the siRNA or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the siRNA (or derivatives thereof). The siRNA (or derivative) is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, 111:206-212 (alpha 1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-gamma and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of siRNA (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified siRNA may also be prepared in different formulations depending on the type of chemical modification or the type of device employed.

Formulations suitable for use with a nebulizer, either jet or ultrasonic, will typically comprise siRNA (or derivative) dissolved in water at a concentration of about 0.1 to 25 mg of biologically active siRNA per mL of solution. The formulation may also include a buffer and a simple sugar (e.g., for siRNA stabilization and regulation of osmotic pressure). The nebulizer formulation may also contain a surfactant, to reduce or prevent surface induced aggregation of the siRNA caused by atomization of the solution in forming the aerosol.

Formulations for use with a metered-dose inhaler device will generally comprise a finely divided powder containing the siRNA (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafiuoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing siRNA (or derivative) and may also include a bulking agent, such as lactose, sorbitol, sucrose, or mannitol in amounts which facilitate dispersal of the powder from the device, e.g., 50 to 90% by weight of the formulation. The siRNA (or derivative) should most advantageously be prepared in particulate form with an average particle size of less than 10 μm (microns), most preferably 0.5 to 5 μm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The siRNA and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of an siRNA and optionally one or more additional therapeutic agents included in a pharmaceutically acceptable carrier.

The therapeutic agent(s), including specifically but not limited to the siRNA, may be provided in particles. Particles as used herein means nano or microparticles (or in some instances larger) which can consist in whole or in part of the siRNA or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the siRNA in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Preparation of Single- and Double-Stranded RNA Species

A series of pairs of synthetic single-stranded oligoribonucleotides (ssORN), selected for use as siRNA derived from sequences of human MAPK2 (Erk2) and Lamin AC genes, were prepared using conventional techniques and reagents. For use as double-stranded siRNA, single-stranded members of each pair were annealed under suitable thermal conditions, followed by isolation using HPLC of double-stranded siRNA from residual ssORN. Sequences are listed in Table 1, where each nucleotide is an unmodified ribonucleotide and each internucleotide linkage is phosphodiester, except as indicated. It will be appreciated that double-stranded siRNA structures included 0-2 unpaired nucleotides (i.e., single-stranded overhangs) at one or both ends.

TABLE 1
RNA Sequences for siRNA

| | | | | |
|---|---|---|---|---|
| | | | | SEQ |
| | | | | ID |
| Target | Name | Strand | Sequence* | NO: |

| | | | | |
|---|---|---|---|---|
| MAPK2 | MAPK2 | s | 5′-UGCUGACUCCAAAGCUCUGTT-3′ | 1 |
| | MAPK2 | as | 5′-CAGAGCUUUGGAGUCAGCATT-3′ | 2 |
| | MAPK2 Exp27 | s | 5′-AAUGCUGACUCCAAAGCUCUGUU-3′ | 3 |
| | MAPK2 Exp27 | as | 5′-CAGAGCUUUGGAGUCAGCAUU-3′ | 4 |
| | MAPK2 Exp30 | s | 5′-AAUGCUGACUCCAAAGCUCUGUU-3′ | 3 |
| | MAPK2 Exp30 | as | 5′-CAGAGCUUUGGAGUCAGCAUU-3′ | 5 |
| Lamin | Lamin AC | s | 5′-CUGGACUUCCAGAAGAACATT-3′ | 6 |
| AC | Lamin AC | as | 5′-UGUUCUUCUGGAAGUCCAGTT-3′ | 7 |
| | Lamin AC Exp27 | s | 5′-AACUGGACUUCCAGAAGAACAUU-3′ | 8 |
| | Lamin AC Exp27 | as | 5′-UGUUCUUCUGGAAGUCCAGUU-3′ | 9 |
| | Lamin AC Exp30 | s | 5′-AACUGGACUUCCAGAAGAACAUU-3′ | 8 |
| | Lamin AC Exp30 | as | 5′-UGUUCUUCUGGAAGUCCAGUU-3′ | 10 |

*Nucleotides and/or internucleotide linkages between nucleotides shown in bold were modified and selected from: 2′ sugar modification as described herein and stabilized linkage between the two 3′-terminal nucleotides.

### Example 2

### Modification of the Sense Strand of Double-Stranded siRNA Inhibits Immunostimulation by siRNA

Human PBMC were isolated from whole blood of healthy individuals by Ficoll-Hypaque density gradient centrifugation. Isolated PBMC were then plated into individual wells of multiwell culture plates in suitable culture medium. Various double-stranded siRNA were added to individual wells over a range of concentration (ca. 2 nM to ca. 0.5 μM), in the presence of DOTAP, and the cells were incubated for 24 h. Culture supernatants were harvested following the incubation and assayed for IFN-alpha and IL-12p40 using suitable ELISA. The various double-stranded siRNA tested were MAPK2, MAPK2 Exp27, MAPK2 Exp30, Lanmin AC, Lamin AC Exp27, and Lamin AC Exp30. Results are shown in FIG. 1. Data are presented as means±SEM.

As shown in FIG. 1, inclusion of nucleotides having 2′ sugar modification in the sense strand of these siRNA strikingly and significantly reduced the amounts of IFN-alpha and, especially, IL-12p40 secreted by PBMC after 24 h incubation with siRNA, compared to control.

### Example 3

### Modification of the Sense Strand of Double-Stranded siRNA is Sufficient to Inhibit Immunostimulation by siRNA

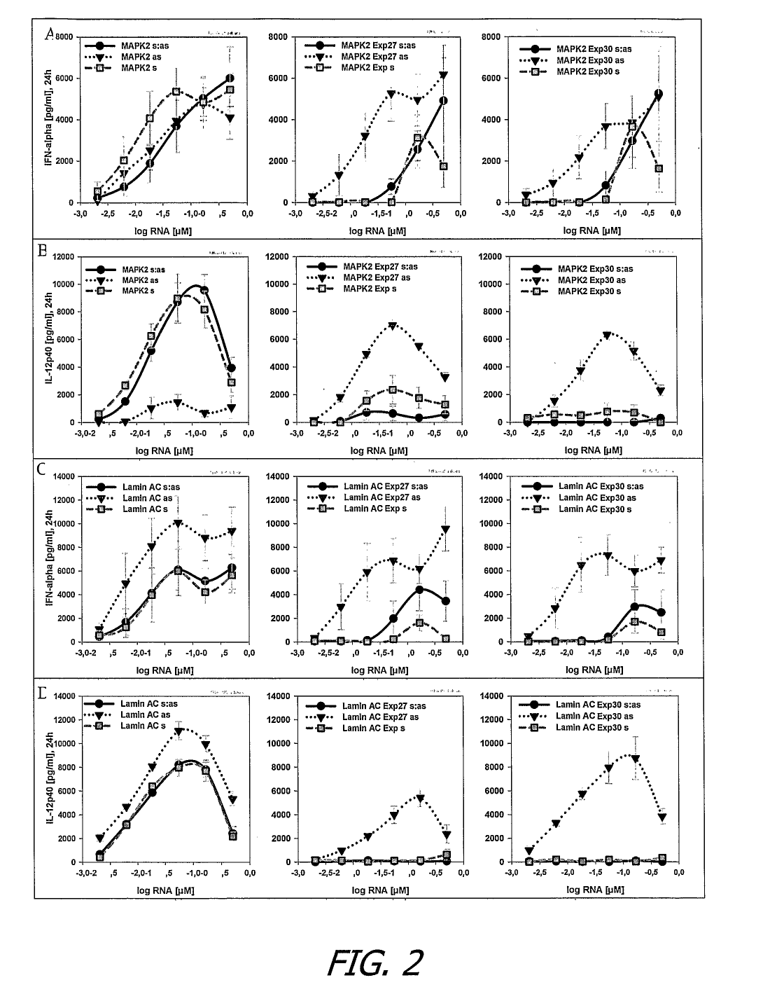

Human PBMC were isolated and plated into multiwell culture plates as in Example 2. Various species of single-stranded and double-stranded RNA were added to individual wells over a range of concentration (ca. 2 nM to ca. 0.5 μM), in the presence of DOTAP, and the cells were incubated for 24 h. Culture supernatants were harvested following the incubation and assayed for IFN-alpha and IL-12p40 using suitable ELISA. Experiments were designed to compare double-stranded (s:as) siRNA to corresponding individual sense and antisense single-stranded RNA. The various double-stranded siRNA tested were MAPK2, MAPK2 Exp27, MAPK2 Exp30, Lamin AC, Lamin AC Exp27, and Lamin AC Exp30. The various single-stranded RNA tested were MAPK2 s, MAPK2 as, MAPK2 Exp27 s, MAPK2 Exp27 as, MAPK2 Exp30s, MAPK2 Exp30 as, Lamin AC s, Lamin AC as, Lamin AC Exp27 s, Lamin AC Exp27 as, Lamin AC Exp30 s, and Lamin AC Exp30 as. Results are shown in FIG. 2. Data are presented as means±SEM.

As shown in FIG. 2, inclusion of nucleotides having 2′ sugar modification in the sense strand of these siRNA strikingly and significantly reduced the amounts of IFN-alpha and, especially, IL-12p40 secreted by PBMC after 24 h incubation with either double-stranded siRNA or single-stranded sense strand alone, compared to control. In contrast, modified antisense strands alone remained strongly immunostimulatory. These same antisense strands, when presented in the context of double-stranded siRNA, however, were far less immunostimulatory, consistent with the notion that modification involving the sense strand alone is necessary and sufficient to reduce the immunostimulatory potential of double-stranded siRNA.

### Example 4

### Modified siRNA with Reduced Immunostimulatory Potential Retain Gene Silencing Properties

In order to assess the gene silencing properties of modified siRNA, human PBMC isolated and cultured as described in Example 2 are assayed for MAPK2 and larnin AC transcripts using quantitative reverse transcriptase-polymerase chain method with suitable primer pairs for each transcript being probed. Transcripts for a housekeeping gene are also measured to normalize measurements. Western blotting and immunocytochemistry are used to confirm corresponding decrease in protein MAPK2 and lamin AC transcript levels are reduced in a dose-dependent manner based on the concentration of siRNA, and, significantly, to similar degree for modified siRNA and corresponding control siRNA.

### Example 5

Other Nucleotide 2′ Sugar Modifications in the Sense Strand of siRNA Reduce Immunostimulation and Preserve Gene Silencing

Additional siRNA are synthesized with any of the following various 2′ sugar modifications of at least one nucleotide in the sense strand of the siRNA: 2′-O-methyl, 2′-deoxy, 2′-fluoro-2′-deoxy, 2′-amino-2′-deoxy, 2′-methoxyethyl (MOE), 2′-O-allyl, 2′-propinyl, 2′-aminopropargyl, 2′-O-(3-aminopropyl), 2′-O-propyl, 2′-O-butyl, or generally 2′-O-alkyl, 2′-O-alkenyl, and 2′-O-alkinyl. In addition, locked nucleic acids (LNA) and arabinosides are used. The various 2′ sugar modifications are introduced in various positions and various numbers along the sense strand. Immunostimulatory and gene silencing effects are assessed in a manner similar to that described in Examples 2-4 above.

### Example 6

### Inclusion of Stabilizing Internucleotide Linkages in Sense Strand

Additional siRNA incorporating any of the various 2′ sugar modifications of at least one nucleotide in the sense strand of the siRNA are synthesized with at least one of any of the following internucleotide linkages in the sense strand: thioformacetal, phosphorothioate, methylphosphonate, boranophosphonate, formacetate, and other dephospho analogs (as described in Uhlmann and Peyman, 1993, Oligonucleotide analogs containing dephospho internucleotide linkages, Methods in Molecular Biology, 20:355, Humana Press, the entire content of which is incorporated by reference herein). The various 2′ sugar and internucleotide linkage modifications are introduced in various positions and various numbers along the sense strand. Immunostimulatory and gene silencing effects are assessed in a manner similar to that described in Examples 2-4 above.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

We claim:

1. A composition comprising a double-stranded short interfering ribonucleic acid (siRNA) having a sense strand and an antisense strand, each strand having a 5′ end and a 3′ end, wherein the antisense strand is complementary to a target sequence and wherein the sense strand comprises at least one modified nucleotide having a sugar with a 2′ modification, with proviso that the modified nucleotide having the sugar with the 2′ modification is not a locked nucleic acid (LNA) or a 2′-O-methyl nucleotide.

2. The composition of claim 1, wherein the sense strand comprises only one modified nucleotide having the sugar with the 2′ modification.

3. The composition of claim 1, wherein the sense strand comprises a plurality of modified nucleotides having the sugar with the 2′ modification, wherein each modified nucleotide having the sugar with the 2′ modification is selected independently of any other.

4. The composition of claim 1, wherein the 2′ modification is selected from the group consisting of 2′-O-alkyl, 2′-O-alkenyl, and 2′-O-alkinyl, with proviso that 2′-O-alkyl excludes 2′-O-methyl.

5. The composition of claim 1, wherein the 2′ modification is selected from the group consisting of 2′-methoxyethyl, 2′-O-allyl, 2′-propinyl, 2′-aminopropargyl, 2′-O-(3-aminopropyl), 2′-O-propyl, and 2′-O-butyl.

6. The composition of claim 1, wherein the 2′ modification is selected from the group consisting of 2′-deoxy, 2′-fluoro, and 2′-amino.

7. The composition of claim 1, wherein the 2′ modification is 2′-fluoro.

8. The composition of claim 1, wherein the 2′ modification is selected from 2′-O-alkenyl, 2′-O-alkinyl, 2′-methoxyethyl, 2′-aminopropargyl, 2′-O-(3-aminopropyl), and 2′-amino.

9. The composition of claim 1, wherein the at least one modified nucleotide having the sugar with the 2′ modification occurs at the 5′ end of the sense strand.

10. The composition of claim 1, wherein the at least one modified nucleotide having the sugar with the 2′ modification occurs at the 3′ end of the sense strand.

11. The composition of claim 1, wherein the at least one modified nucleotide having the sugar with the 2′ modification occurs internal with respect to the 5′ end and the 3′ end of the sense strand.

12. The composition of claim 1, wherein the sense strand comprises at least one modified nucleotide having the sugar with the 2′ modification at the 5′ end of the sense strand and at least one modified nucleotide having the sugar with the 2′ modification at the 3′ end of the sense strand.

13. The composition of claim 1, wherein the sense strand has a phosphodiester backbone.

14. The composition of claim 1, wherein the sense strand has a stabilized backbone comprising at least one stabilized internucleotide linkage.

15. The composition of claim 1, wherein the sense strand has a stabilized backbone comprising at least one stabilized internucleotide linkage selected from the group consisting of thioformacetal, phosphorothioate, methylphosphonate, boranophosphonate, and formacetate.

16. A method for reducing immunostimulatory potential of a double-stranded short interfering ribonucleic acid (siRNA), said siRNA having a sense strand and an antisense strand, each strand having a 5′ end and a 3′ end, wherein the antisense strand is complementary to a target sequence, the method comprising introducing into the sense strand of the siRNA at least one modified nucleotide having a sugar with a 2′ modification, with proviso that the modified nucleotide having the sugar with the 2′ modification is not a locked nucleic acid (LNA) or a 2′-O-methyl nucleotide.

17. The method of claim 16, wherein the introducing is introducing only one modified nucleotide having the sugar with the 2′ modification.

18. The method of claim 16, wherein the introducing is introducing a plurality of modified nucleotides having the sugar with the 2′ modification, wherein each modified nucleotide having the sugar with the 2′ modification is selected independently of any other.

19. The method of claim 16, wherein the 2′ modification is selected from the group consisting of 2′-O-alkyl, 2′-O-alkenyl, and 2′-O-alkinyl, with proviso that 2′-O-alkyl excludes 2′-O-methyl.

20. The method of claim 16, wherein the 2′ modification is selected from the group consisting of 2′-methoxyethyl, 2′-O-allyl, 2′-propinyl, 2′-aminopropargyl, 2′-O-(3-aminopropyl), 2′-O-propyl, and 2′-O-butyl.

21. The method of claim 16, wherein the 2′ modification is selected from the group consisting of 2′-deoxy, 2′-fluoro, and 2′-amino.

22. The method of claim 16, wherein the 2′ modification is 2′-fluoro.

23. The method of claim 16, wherein the 2′ modification is selected from 2′-O-alkenyl, 2′-O-alkinyl, 2′-methoxyethyl, 2′-aminopropargyl, 2′-O-(3-aminopropyl), and 2′-amino.

24. The method of claim 16, wherein the introducing occurs at the 5′ end of the sense strand.

25. The method of claim 16, wherein the introducing occurs at the 3′ end of the sense strand.

26. The method of claim 16, wherein the introducing occurs internal with respect to the 5′ end and the 3′ end of the sense strand.

27. The method of claim 16, wherein the introducing occurs at the 5′ end of the sense strand and at the 3′ end of the sense strand.

28. The method of claim 16, wherein the sense strand has a phosphodiester backbone.

29. The method of claim 16, wherein the sense strand has a stabilized backbone comprising at least one stabilized internucleotide linkage.

30. The method of claim 16, wherein the sense strand has a stabilized backbone comprising at least one stabilized internucleotide linkage selected from the group consisting of thioformacetal, phosphorothioate, methylphosphonate, boranophosphonate, and formacetate.

31. A method for reducing expression of a gene having a target sequence, the method comprising contacting a cell comprising the gene having the target sequence with an effective amount of a double-stranded short interfering ribonucleic acid (siRNA) having a sense strand and an antisense strand, each strand having a 5′ end and a 3′ end, wherein the antisense strand is complementary to the target sequence and wherein the sense strand comprises at least one modified nucleotide having a sugar with a 2′ modification, with proviso that the modified nucleotide having the sugar with the 2′ modification is not a locked nucleic acid (LNA) or a 2′-O-methyl nucleotide, to reduce expression of the gene having the target sequence.

32. The method of claim 31, wherein the sense strand comprises only one modified nucleotide having the sugar with the 2′ modification.

33. The method of claim 31, wherein the sense strand comprises a plurality of modified nucleotides having the sugar with the 2′ modification, wherein each modified nucleotide having the sugar with the 2′ modification is selected independently of any other.

34. The method of claim 31, wherein the 2′ modification is selected from the group consisting of 2′-O-alkyl, 2′-O-alkenyl, and 2′-O-alkinyl, with proviso that 2′-O-alkyl excludes 2′-O-methyl.

35. The method of claim 31, wherein the 2′ modification is selected from the group consisting of 2′-methoxyethyl, 2′-O-allyl, 2′-propinyl, 2′-aminopropargyl, 2′-O-(3-aminopropyl), 2′-O-propyl, and 2′-O-butyl.

36. The method of claim 31, wherein the 2′ modification is selected from the group consisting of 2′-deoxy, 2′-fluoro, and 2′-amino.

37. The method of claim 31, wherein the 2′ modification is 2′-fluoro.

38. The method of claim 31, wherein the 2′ modification is selected from 2′-O-alkenyl, 2′-O-alkinyl, 2′-methoxyethyl, 2′-aminopropargyl, 2′-O-(3-aminopropyl), and 2′-amino.

39. The method of claim 31, wherein the at least one modified nucleotide having the sugar with the 2′ modification occurs at the 5′ end of the sense strand.

40. The method of claim 31, wherein the at least one modified nucleotide having the sugar with the 2′ modification occurs at the 3′ end of the sense strand.

41. The method of claim 31, wherein the at least one modified nucleotide having the sugar with the 2′ modification occurs internal with respect to the 5′ end and the 3′ end of the sense strand.

42. The method of claim 31, wherein the sense strand comprises at least one modified nucleotide having the sugar with the 2′ modification at the 5′ end of the sense strand and at least one modified nucleotide having the sugar with the 2′ modification at the 3′ end of the sense strand.

43. The method of claim 31, wherein the sense strand has a phosphodiester backbone.

44. The method of claim 31, wherein the sense strand has a stabilized backbone comprising at least one stabilized internucleotide linkage.

45. The method of claim 31, wherein the sense strand has a stabilized backbone comprising at least one stabilized internucleotide linkage selected from the group consisting of thioformacetal, phosphorothioate, methylphosphonate, boranophosphonate, and formacetate.
